(19) United States
(12) Patent Application Publication
(10) Pub. No.: US 2007/0270393 A1
(43) Pub. Date: Nov. 22, 2007

(54) Methods and compositions for modulation of sleep cycle

(76) Inventors: Theresa Buckley, Palo Alto, CA (US); Alan F. Schatzberg, Los Altos, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/707,751

(22) Filed: Feb. 15, 2007

Related U.S. Application Data
(60) Provisional application No. 60/774,355, filed on Feb. 17, 2006.

(51) Int. Cl. A61K 31/56; A61P 43/00

(52) U.S. Cl. 514/171

(57) ABSTRACT

Methods and compositions for modulating the circadian rhythm of a subject are provided. In practicing the subject methods, modulation of the circadian rhythm of a subject is achieved by administering to the subject an effective amount of at least one of a glucocorticoid receptor antagonist, a CRH antagonist and an MR agonist. Also provided are kits for practicing the subject methods.

### CROSS-REFERENCE TO RELATED APPLICATIONS

Pursuant to 35 U.S.C. §119(e), this application claims priority to the filing date of the U.S. Provisional Patent Application Ser. No. 60/774,355 filed Feb. 17, 2006; the disclosure of which provisional applications is herein incorporated by reference.

### GOVERNMENT RIGHTS

This invention was made with government support under federal grant nos. MH19938 awarded by National Institute of Mental Health. The United States Government may have certain rights in this invention.

### INTRODUCTION

It is well established that living organisms have internal biological clocks which regulate activities such as their sleep/wake cycles. These biological clocks are expressions of the effects of one or more endogenous pacemakers thought to be located in the suprachiasmatic nuclei (SCN) of the hypothalamus. Biological rhythms are repetitive fluctuations in various biological signals over time, and are often driven by the biological clocks. A circadian rhythm occurs when the period of that fluctuation is about a day or about 24 hours. A circadian rhythm can manifest in terms of numerous biological variables and measures. Examples include the circadian rhythm to alertness, the circadian rhythm to core body temperature, the circadian rhythm to certain hormone production, the circadian rhythm to blood pressure, the circadian rhythm to activity, the circadian rhythm of wake/sleep cycles, etc. Examples of hormonal circadian rhythms include those of cortisol, CRH and melatonin.

Different organisms have different activity cycles. Creatures which tend to be active during periods of daylight and inactive at night are termed diurnal. Creatures which are active at night and sleep during the day are referred to as nocturnal. In general, the phase or timing of natural circadian rhythms are entrained by and tend to follow the natural sequence of daytime light and nighttime darkness which occurs as the earth rotates. Hence light has a chronobiotic effect, or phase shifting effect on the circadian rhythm and can assist with synchronization of the timing of the circadian rhythm.

On the other hand, there may be certain shapes to the waveform of each of these different circadian rhythms. For example,.some waveforms may be sinusoidal, linear, or a combination of several superimposed waveforms. In some cases, it may be useful to adjust the shape of the circadian waveform to assist with nocturnal sleep. In some cases, the amplitude of the circadian rhythm amplitude may be flattened, its magnitude at the nadir may be increased, the entire waveform may be elevated in magnitude, the time between acrophase and nadir may be shortened or prolonged, etc.

Under normal circumstances, the circadian rhythm of humans serves as a useful time regulator of various activities. However, some persons suffer from circadian rhythm phase irregularities such as advanced or Circadian Rhythm Sleep Disorder, Delayed Sleep Phase Type which such disorders result in an interference with maintenance of normal activity pattern. As a result such persons suffering from circadian rhythm irregularities and disorders experience disruptions in their sleep patterns.

On the other hand, some suffer from disorders associated with alterations in the shape of the waveform of certain circadian rhythms. For example, in insomnia, the shape of the waveform of the circadian rhythm of cortisol may be elevated at certain times of night, compared to controls (Vgontzas et al., 2001, J. Clin. Endocrinol. Metabl., 86(4):1489-1495; and Rodenbeck and Hajak, J Clin Psychiatry. 2001 June; 62(6):453-63). An elevated nocturnal cortisol or ACTH at the nadir may be a marker for increased HPA activity at night. As a result, such circadian waveform irregularities may disrupt sleep patterns.

In addition, in other instances, the internal circadian clock interferes with desired adaptations to differing time schedules. For example, air travelers who rapidly cross two or more time zones may find their internal circadian clocks out of phase with the day/night cycle at their destination, giving rise to the so-called “jet-lag” syndrome in which they suffer disruptions of their sleep patterns and diminished attention span and alertness until their inner biological clocks gradually adjust to local time. Shift workers, whose work schedules rotate among day shift, night shift and the so-called “graveyard” shift, may experience transient internal temporal dissociation or a lack of synchronization among various bodily rhythms, and consequent difficulty in adjusting to shift changes. This can adversely affect worker productivity, and in some instances may raise safety concerns.

A general need exists for the regulation and control of the circadian rhythm of a subject, Regulation and control of the circadian rhythm of a subject would be beneficial for a number of diseases or disorders related to circadian rhythm irregularities, where control of the circadian rhythm will contribute to treatment of a sleep disorder. In addition, control of the circadian rhythm of a subject would also be beneficial in instances in which the internal circadian rhythm interferes with desired adaptations to differing time schedules. Accordingly, there continues to be a need for development of such methods.

### SUMMARY

Methods and compositions for modulating the circadian rhythm of a subject are provided. In practicing the subject methods, modulation of the circadian rhythm of a subject is achieved by administering to the subject an effective amount of at least one of a glucocorticoid receptor antagonist, a CRH antagonist and an MR agonist. Also provided are kits for practicing the subject methods.

Aspects of the invention include a method for modulating a circadian rhythm of a subject by administering to the subject an effective amount of a at least one of glucocorticoid receptor antagonist, a CRH antagonist and an MR agonist to modulate the circadian rhythm in the subject. In one embodiment, the modulating results in phase shifting a circadian rhythm to improve the subject's sleep. In another embodiment, the modulating results in a change in the shape of the circadian waveform to improve the subject's sleep. In another embodiment, the method is a method for treating a subject for a sleep disorder. In such embodiments the sleep disorder is insomnia, Circadian Rhythm Sleep Disorder, Delayed Sleep Phase Type, Circadian Rhythm Sleep Disorder, Advanced Sleep Phase Type, Circadian Rhythm Sleep Disorder, Irregular Sleep-Wake Type, Circadian Rhythm Sleep Disorder, Free-Running Type, short sleeper, long sleeper, obstructive sleep apnea. In other embodiments the method is a method for treating inter-time zone travel induced sleep disturbance (Circadian Rhythm Sleep Disorder, Jet Lag Type), e.g., Circadian Rhythm Sleep Disorder, Jet Lag Type. In yet other embodiments the method is a method for treating Circadian Rhythm Sleep Disorder, Shift Work Type. In certain embodiments the subject is a mammal, such as a human.

In certain embodiments where the agent employed is a glucocorticoid receptor antagonist, the glucocorticoid receptor antagonist includes a steroidal skeleton with at least one phenyl-containing moiety in the 11-beta position of the steroidal skeleton. In further embodiments the phenyl-containing moiety in the 11-beta position of the skeleton is a dimethylaminophenyl moiety. In yet further embodiments, the glucocorticoid receptor antagonist is mifepristone or is selected from the group consisting of RU009 and RU044.

Aspects of the invention further include methods for treating a sleep disorder in a mammalian subject including administering to the subject an effective amount of at least one of a glucocorticoid receptor antagonist, a CRH antagonist and an MR agonist to treat the sleep disorder in the subject. In some embodiments, the sleep disorder is insomnia. In other embodiments the sleep disorder is delayed sleep phase syndrome. In yet other embodiments the sleep disorder is Circadian Rhythm Sleep Disorder, Advanced Sleep Phase Type. In other embodiments the sleep disorder is shift-work sleep disorder. In other embodiments the sleep disorder is Circadian Rhythm Sleep Disorder, Jet Lag Type. In other embodiments the sleep disorder is Circadian Rhythm Sleep Disorder, Irregular Sleep-Wake Type. In other embodiments the sleep disorder is Circadian Rhythm Sleep Disorder, Free-Running Type. In other embodiments the sleep disorder is short sleeper. In other embodiments, the sleep disorder is long sleeper. In other embodiments the sleep disorder is obstructive sleep apnea. In such embodiments the subject may be a human.

Where employed, in certain embodiments the glucocorticoid receptor antagonist includes a steroidal skeleton with at least one phenyl-containing moiety in the 11-beta position of the steroidal skeleton. In further embodiments the phenyl-containing moiety in the 11-beta position of the skeleton is a dimethylaminophenyl moiety. In yet further embodiments, the glucocorticoid receptor antagonist is mifepristone or is selected from the group consisting of RU009 and RU044.

Yet another feature of the present invention is a method for modulating phase shifting of a circadian rhythm to accommodate an environmentally imposed desired sleep cycle of a mammalian subject including administering to the subject an effective amount of at least one of a glucocorticoid receptor antagonist, a CRH antagonist and an MR agonist to modulate phase shifting of the sleep cycle of the subject. In some embodiments, the method is a method for treating a subject for a Circadian Rhythm Sleep Disorder, Shift Work Type. In other embodiments, the method is a method for treating a subject for inter-time zone travel induced sleep disturbance (Circadian Rhythm Sleep Disorder, Jet Lag Type). In such embodiments the subject is a human.

Yet another aspect of the invention is a method for modulating a relative phase shift between two or more circadian rhythms, including between cortisol and melatonin.

In certain embodiments the glucocorticoid receptor antagonist includes a steroidal skeleton with at least one phenyl-containing moiety in the 11-beta position of the steroidal skeleton. In further embodiments the phenyl-containing moiety in the 11-beta position of the skeleton is a dimethylaminophenyl moiety. In yet further embodiments, the glucocorticoid receptor antagonist is mifepristone or is selected from the group consisting of RU009 and RU044.

Yet another aspect of the present invention is a method of treating a sleep disorder in a subject including identifying a subject suffering from a sleep disorder; and administering to the subject an effective amount of at least one of a glucocorticoid receptor antagonist, a CRH antagonist and an MR agonist to treat the subject for the sleep disorder. In some embodiments, the sleep disorder is insomnia. In other embodiments, the sleep disorder is Circadian Rhythm Sleep Disorder, Delayed Sleep Phase Type. In yet other embodiments, the sleep disorder is Circadian Rhythm Sleep Disorder, Advanced Sleep Phase Type. In other embodiments the sleep disorder is shift-work sleep disorder. In other embodiments the sleep disorder is an inter-time zone travel induced sleep disturbance (Circadian Rhythm Sleep Disorder, Jet Lag Type), e.g., Circadian Rhythm Sleep Disorder, Jet Lag Type. In other embodiments the sleep disorder is Circadian Rhythm Sleep Disorder, Irregular Sleep-Wake Type. In other embodiments the sleep disorder is Circadian Rhythm Sleep Disorder, Free-Running Type. In other embodiments the sleep disorder is short sleeper. In other embodiments, the sleep disorder is long sleeper. In other embodiments the sleep disorder is obstructive sleep apnea. In such embodiments the subject is a mammal, such as a human.

Yet another aspect of the present invention is a method of treating the metabolic or other endocrine consequences of a sleep disorder. Complications may include hypercortisolemia (i.e., as in sleep apnea) that may contribute to insomnia. Other endocrine complications of a sleep disorder, such as obstructive sleep apnea, include metabolic complications such as hypercortisolemia induced insulin resistance.

In certain embodiments the glucocorticoid receptor antagonist includes a steroidal skeleton with at least one phenyl-containing moiety in the 11-beta position of the steroidal skeleton. In further embodiments the phenyl-containing moiety in the 11-beta position of the skeleton is a dimethylaminophenyl moiety. In yet further embodiments, the glucocorticoid receptor antagonist is mifepristone or is selected from the group consisting of RU009, RU044 ORG-34517, ORG-34850, and ORG-34116.

Aspects of the present invention further include kits having a at least one of a glucocorticoid receptor antagonist, CRH antagonist and MR agonist; and instructions for using the agent(s) to treat a sleep disorder in a subject. In such embodiments where the agent is a glucocorticoid receptor antagonist, the glucocorticoid receptor antagonist includes a steroidal skeleton with at least one phenyl-containing moiety in the 11-beta position of the steroidal skeleton. In further embodiments the phenyl-containing moiety in the 11-beta position of the skeleton is a dimethylaminophenyl moiety. In yet further embodiments, the glucocorticoid receptor antagonist is mifepristone or is selected from the group consisting of RU009 and RU044.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features may be arbitrarily expanded or reduced for clarity. Included in the drawings are the following figures:

FIG. 1 is a graph showing relative change in polysomnogram measures of treatment group to placebo group. The scores show the difference between five days following treatment with mifepristone compared to baseline and two weeks following discontinuation of mifepristone administration compared to baseline. Polysomnogram abbreviations: WASO=wake after sleep onset; % S1=percentage stage 1 sleep; % S2=percentage stage 2 sleep; % S3=percentage stage 3 sleep; % S4=percentage stage 4 sleep; % REM=percentage REM sleep; NUAW=number of awakenings; TST=total combined minutes of sleep; S3 Lat (min)=latency to onset of stage 3 sleep; SEB=total combined minutes of stage 2, 3 and 4 per total sleep minutes; SEC=total combined minutes of stage 3, 4 per total sleep minutes; % NEM=sum of % S3 and % S4; REM lat=minutes to first epoch of REM after sleep onset; REM density=# rapid eye movements per minutes of REM sleep; and Arousal index=computerized measure high frequency bands/minutes sleep.

FIG. 2 is a graph showing absolute change in polysomnogram measures of treatment group. The scores show the difference between five days following treatment with mifepristone compared to baseline and two weeks following discontinuation of mifepristone administration compared to baseline. Abbreviations are the same as in FIG. 1.

FIG. 3 is a series of graphs showing overnight melatonin levels in dim lights in chronic insomnia subjects that have been administered either a placebo (Panel A) or mifepristone (Panel B). The graphs provide time points for measurements prior to administration, five days following administration, and two weeks following discontinuation of administration of either placebo (Panel A) or mifepristone (Panel B).

FIG. 4 is a series of graphs showing overnight cortisol (Panel A) and ACTH (Panel B) levels in chronic insomnia subjects administered either mifepristone or placebo. The longitudinal date of the graphs is divided into three sections: first section=prior to treatment; second section=−five days following administration; third section=two weeks following discontinuation of administration.

FIG. 5 is a graph showing overnight cortisol levels in healthy control subjects that have been administered mifepristone. Diamonds show measurements prior to administration of mifepristone and squares show measurements two days after administration of mifepristone.

FIGS. 6 to 8 provide graphs of results from various studies reported in Example 3, below.

### DEFINITIONS

The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; or improving a subject's physical or mental well-being. The treatment or amelioration of symptoms can be based on objective or subjective parameters; including the results of a physical examination and/or evaluation of sleep patterns.

The term “glucocorticoid receptor antagonist” refers to any composition or compound which partially or completely inhibits (antagonizes) the binding of a glucocorticoid receptor (GR) agonist, such as cortisol, or cortisol analogs, synthetic or natural, to a GR. A “glucocorticoid receptor antagonist” also refers to any composition or compound which inhibits any biological response associated with the binding of a GR to an agonist.

The term “glucocorticoid receptor” (“GR”) refers to a family of intracellular receptors also referred to as the cortisol receptor, which specifically bind to cortisol and/or cortisol analogs. The term includes isoforms of GR, recombinant GR and mutated GR.

The term “cortisol” refers to a family of compositions also referred to hydrocortisone, and any synthetic or natural analogues thereof.

The term “mifepristone” refers to a family of compositions also referred to as RU486, or RU38.486, or 17-beta-hydroxy-11-beta-(4-dimethyl-aminophenyl)-17-alpha-(1-propynyl)-estra-4,9-dien-3-one), or 11-beta-(4dimethylaminophenyl)-17-beta-hydroxy-17-alpha-(1-propynyl)-estra-4,9-dien-3-one), or analogs thereof, which bind to the glucocorticoid receptor, typically with high affinity, and inhibit the biological effects initiated/mediated by the binding of any cortisol or cortisol analogue to a receptor. Chemical names for RU-486 vary; for example, RU486 has also been termed: 11B-[p-(Dimethylamino)phenyl)-17B-hydroxy-17-(1-propynyl)-estra-4,9-dien-3-one; 11B-(4-dimethyl-aminophenyl)-17B-hydroxy-17A-(prop-1-ynyl)-estra-4,9-dien-3-one; 17B-hydroxy-11B-(4-dimethylaminophenyl-1)-17A-(propynyl-1)-estra-4,9-diene-3-one; 17B-hydroxy-11B-(4-dimethylaminophenyl-1)-17A-(propynyl-1)-E; (11B,17B)-11[4-dimethylamino)-phenyl]-17-hydroxy-17-(1-propynyl)estra-4,9-dien-3-one; and 11B-[4-(N,N-dimethylamino)phenyl]-17A-(prop-1-ynyl)-D-4,9-estradiene-17B-ol-3-one.

### DETAILED DESCRIPTION

Methods and compositions for modulating the circadian rhythm of a subject are provided. In practicing the subject methods, modulation of the circadian rhythm of a subject is achieved by administering to the subject an effective amount of a glucocorticoid receptor antagonist. Also provided are kits for practicing the subject methods.

Before the present invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Unless defined otherwise, all technical and scientific terms used herein have the same meaning as commonly understood to one of ordinary skill in the art to which this invention belongs.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the invention, representative methods are discussed first in greater detail, followed by a review of representative applications for the methods, as well as kits that find use in practicing the subject methods.

Methods

As summarized above, the subject invention provides a method for modulating a circadian rhythm of a subject. By “circadian rhythm” is meant the daily, i.e., 24 hour, Hypothalamic-Pituitary Adrenal (HPA) axis cycle of cortisol, Adrenocorticotropic Hormone (ACTH) or corticotropin-releasing hormone (CRH), melatonin, core body temperature. By “circadian rhythm of the activity cycle” is meant the 24 hour cycle of activity and rest patterns. By “circadian rhythm of a sleep cycle” is meant the 24 hour cycle of sleep versus wake state, where a normal sleep cycle is characterized by six states, consisting of an awake stage (stage W), and sleep stage, which stage includes light sleep (stages 1 and 2), slow wave sleep (stages 3 and 4) and rapid-eye movement sleep (REM) stage. During such a normal sleep cycle, the greatest portion of slow-wave sleep (SWS) occurs in the first part of the night, and the greatest portion of REM occurs in the second half of the night. As used herein “modulating” means changing, including delaying and progressing, the waveform shape, including the amplitude, etc., of the circadian rhythm of a subject. As such, in some embodiments the modulating may result in phase-advancing the circadian rhythm, wherein the circadian rhythm of the sleep cycle is advanced from an awake state to a sleep state. -In other embodiments the modulating may result in phase-delaying the circadian rhythm, wherein the circadian rhythm of the sleep cycle is delayed from progressing from an awake state to a sleep state.

In such embodiments, phase shifting, e.g., phase-advancement or phase-delay, in the circadian rhythm can be monitored based on measurements of the subject's circadian markers that can reasonably predict the endogenous circadian pacemaker. Such circadian markers include, but are not limited to, the following: body core temperature, which decreases as the sleep cycle advances from the awake stage to the sleep stage, sleep latency (i.e., the time it takes to fall asleep), natural wake time, timing of the nadir of cortisol production, timing of the acrophase of cortisol production, timing of the Dim Light Melatonin Onset (DLMO), timing of DIM Light Melatonin Offset, timing of onset of REM latency relative to sleep onset (REM latency). In such methods, the phase of a circadian rhythm of a subject is modulated by administering to the subject an effective amount of a glucocorticoid receptor antagonist in a manner sufficient to modulate the circadian rhythm of the subject.

In other embodiments, the modulating effect results in adjusting the shape of the circadian rhythm waveform to improve sleep. In such methods, the waveform shape can be monitored or measured by other characteristics. Such characteristics include, but are not limited to, height of nadir, amplitude of waveform, height of acrophase, mesor, distance between the acrophase and nadir, area under the curve.

A variety of subjects are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects will be humans.

The invention provides for methods of modulating a circadian rhythm of a subject by administering to the subject an effective amount of at least one of a glucocorticoid receptor (GR) antagonist, a CRH antagonist and a MR agonist to modulate the circadian rhythm of the subject. In certain embodiments, only one of these agents is employed. In certain embodiments, two or these agents are employed. In certain embodiments, three of these agents are employed. Antagonists of GR activity utilized in embodiments of the methods of the invention are well described in the scientific and patent literature. A few illustrative examples are set forth below. Likewise, CRH antagonists utilized in embodiments of the methods of the invention are well described in the scientific and patent literature. A few illustrative examples are set forth below. Likewise, MR agonists utilized in embodiments of the methods of the invention are well described in the scientific and patent literature. A few illustrative examples are set forth below.

Suitable for use in embodiments of the subject methods are steroidal anti-glucocorticoids as GR antagonists. Steroidal antiglucocorticoids can be obtained by modification of the basic structure of glucocorticoid agonists, i.e., varied forms of the steroid backbone. The structure of cortisol, which binds the GR, can be modified in a variety of ways. The two most commonly known classes of structural modifications of the cortisol steroid backbone to create glucocorticoid antagonists include modifications of the 11-beta hydroxy group and modification of the 17-beta side chain (see, e.g., Lefebvre (1989) J. Steroid Biochem. 33:557-563).

Glucocorticoid agonists with modified steroidal backbones comprising removal or substitution of the 11-beta hydroxy group are administered in one embodiment of the invention. This class includes natural antiglucocorticoids, including cortexolone, progesterone and testosterone derivatives, and synthetic compositions, such as mifepristone (Lefebvre, et al. (1989) Ibid). An exemplary embodiment of the invention includes all 11-beta-aryl steroid backbone derivatives because these compounds are devoid of progesterone receptor (PR) binding activity (Agarwal (1987) FEBS 217:221-226). Another exemplary embodiment comprises an 11-beta phenyl-aminodimethyl steroid backbone derivative, i.e., mifepristone, which is both an effective anti-glucocorticoid and anti-progesterone agent. These compositions act as reversibly-binding steroid receptor antagonists. For example, when bound to a 1-beta phenyl-aminodimethyl steroid, the steroid receptor is maintained in a conformation that cannot bind its natural ligand, such as cortisol in the case of GR (Cadepond (1997), supra).

Synthetic 11-beta phenyl-aminodimethyl steroids include mifepristone, also known as RU486, or 17-beta-hydrox-11-beta-(4-dimethyl-aminophenyl) 17-alpha-(1-propynyl)estra-4,9-dien-3-one). Mifepristone has been shown to be a powerful antagonist of both the progesterone and glucocorticoid (GR) receptors. Another 11-beta phenyl-aminodimethyl steroids shown to have GR antagonist effects includes RU009 (RU39.009), 11-beta-(4-dimethyl-aminoethoxyphenyl)-17-alpha-(propynyl-17beta-hydroxy-4,9-estradien-3-one) (see Bocquel (1993) J. Steroid Biochem. Molec. Biol. 45:205-215). Another GR antagonist related to RU486 is RU044 (RU43.044) 17-beta-hydrox-17-alpha-19-(4-methyl-phenyl)-androsta-4,9(11)-dien-3-one) (Bocquel (1993) supra). See also Teutsch (1981) Steroids 38:651-665; U.S. Pat. Nos. 4,386,085 and 4,912,097. Other exemplary glucocorticoid receptor antagonists include, but are not limited to, ORG-34517, ORG-34850, and ORG-34116.

One embodiment includes compositions containing the basic glucocorticoid steroid structure which are irreversible anti-glucocorticoids. Such compounds include alpha-keto-methanesulfonate derivatives of cortisol, including cortisol-21-mesylate (4-pregnene-11-beta, 17-alpha, 21-triol-3,20-dione-21-methane-sulfonate and dexamethasone-21-mesylate (16-methyl-9alpha-fluoro-1,4-pregnadiene-11beta, 17-alpha, 21-triol-3,20-dione-21-methane-sulfonate). See Simons (1986) J. Steroid Biochem. 24:25-32 (1986); Mercier (1986) J. Steroid Biochem. 25:11-20; U.S. Pat. No. 4,296,206.

Steroidal antiglucocorticoids which can be obtained by various structural modifications of the 17-beta side chain are also used in the methods of the invention. This class includes synthetic antiglucocorticoids such as dexamethasone-oxetanone, various 17,21-acetonide derivatives and 17-beta-carboxamide derivatives of dexamethasone (Lefebvre (1989) supra; Rousseau (1979) Nature 279:158-160).

GR antagonists used in the various embodiments of the invention include any steroid backbone modification which effects a biological response resulting from a GR-agonist interaction. Steroid backbone antagonists can be any natural or synthetic variation of cortisol, such as adrenal steroids missing the C-19 methyl group, such as 19-nordeoxycorticosterone and 19-norprogesterone (Wynne (1980) Endocrinology 107:1278-1280).

In general, the 11-beta side chain substituent, and particularly the size of that substituent, can play a key role in determining the extent of a steroid's antiglucocorticoid activity. Substitutions in the A ring of the steroid backbone can also be important. 17-hydroxypropenyl side chains generally decrease antiglucocorticoidal activity in comparison to 17-propinyl side chain containing compounds.

Non-steroidal glucocorticoid antagonists are also used in the subject methods of the invention. These include synthetic mimetics and analogs of proteins, including partially peptidic, pseudopeptidic and non-peptidic molecular entities. For example, oligomeric peptidomimetics useful in the invention include (alpha-beta-unsaturated) peptidosulfonamides, N-substituted glycine derivatives, oligo carbamates, oligo urea peptidomimetics, hydrazinopeptides, oligosulfones and the like (see, e.g., Amour (1994) Int. J. Pept. Protein Res. 43:297-304; de Bont (1996) Bioorganic & Medicinal Chem. 4:667-672). The creation and simultaneous screening of large libraries of synthetic molecules can be carried out using well-known techniques in combinatorial chemistry, for example, see van Breemen (1997) Anal Chem 69:2159-2164; Lam (1997) Anticancer Drug Des 12:145-167 (1997). Design of peptidomimetics specific for GR can be designed using computer programs in conjunction with combinatorial chemistry (combinatorial library) screening approaches (Murray (1995) J. of Computer-Aided Molec. Design 9:381-395); Bohm (1996) J. of Computer-Aided Molec. Design 10:265-272). Such “rational drug design” can help develop peptide isomerics and conformers including cycloisomers, retro-inverso isomers, retro isomers and the like (as discussed in Chorev (1995) Tib Tech 13:438-445).

Because any GR antagonist can be used for the subject methods of the invention, in addition to the compounds and compositions described above, additional useful GR antagonists can be determined by the skilled artisan. A variety of such routine, well-known methods can be used and are described in the scientific and patent literature. They include in vitro and in vivo assays for the identification of additional GR antagonists. A few illustrative examples are described below.

One assay that can be used to identify a GR antagonist of the invention measures the effect of a putative GR antagonist on tyrosine amino-transferase activity in accordance with the method of Granner (1970) Meth. Enzymol. 15:633. This analysis is based on measurement of the activity of the liver enzyme tyrosine amino-transferase (TAT) in cultures of rat hepatoma cells (RHC). TAT catalyzes the first step in the metabolism of tyrosine and is induced by glucocorticoids (cortisol) both in liver and hepatoma cells. This activity is easily measured in cell extracts. TAT converts the amino-group of tyrosine to 2-oxoglutaric acid. P-hydroxyphenylpyruvate is also formed. It can be converted to the more stable p-hydroxybenzaldehyde in an alkaline solution and quantitated by absorbance at 331 nm. The putative GR antagonist is co-administered with cortisol to whole liver, in vivo or ex vivo, or hepatoma cells or cell extracts. A compound is identified as a GR antagonist when its administration decreases the amount of induced TAT activity, as compared to control (i.e., only cortisol or GR agonist added) (see also Shirwany (1986) “Glucocorticoid regulation of hepatic cytosolic glucocorticoid receptors in vivo and its relationship to induction of tyrosine aminotransferase,” Biochem. Biophys. Acta 886:162-168).

Further illustrative of the many assays which can be used to identify compositions utilized in the methods of the invention, in addition to the TAT assay, are assays based on glucocorticoid activities in vivo. For example, assays that assess the ability of a putative GR antagonist to inhibit uptake of ³H-thymidine into DNA in cells which are stimulated by glucocorticoids can be used. Alternatively, the putative GR antagonist can complete with ³H-dexamethasone for binding to a hepatoma tissue culture GR (see, e.g., Choi (1992) “Enzyme induction and receptor-binding affinity of steroidal 20-carboxamides in rat hepatoma tissue culture cells,” Steroids 57:313-318). As another example, the ability of a putative GR antagonist to block nuclear binding of ³H-dexamethasone-GR complex can be used (Alexandrova (1992) “Duration of antagonizing effect of RU486 on the agonist induction of tyrosine aminotransferase via glucocorticoid receptor,” J. Steroid Biochem. Mol. Biol. 41:723-725). To further identify putative GR antagonists, kinetic assays able to discriminate between glucocorticoid agonists and antagonists by means of receptor-binding kinetics can also be used (as described in Jones (1982) Biochem J. 204:721-729).

In another illustrative example, the assay described by Daune (1977) Molec. Pharm. 13:948-955, and in U.S. Pat. No. 4,386,085, can be used to identify anti-glucocorticoid activity. Briefly, the thymocytes of surrenalectomized rats are incubated in nutritive medium containing dexamethasone with the test compound (the putative GR antagonist) at varying concentrations. ³H-uridine is added to the cell culture, which is further incubated, and the extent of incorporation of radiolabel into polynucleotide is measured. Glucocorticoid agonists decrease the amount of ³H-uridine incorporated. Thus, a GR antagonist will oppose this effect.

For additional compounds that can be utilized in the methods of the invention and methods of identifying and making such compounds, see U.S. Pat. No. 4,296,206 (see above); U.S. Pat. No. 4,386,085 (see above); U.S. Pat. Nos. 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; and 5,616,458; and WO 96/19458, which describes non-steroidal compounds which are high-affinity, highly selective modulators (antagonists) for steroid receptors, such as 6-substituted-1,2-dihydro N-1 protected quinolines.

Antiglucocorticoids, such as mifepristone, are formulated as pharmaceuticals to be used in the subject methods of the invention. Routine means to determine GR antagonist drug regimens and formulations to practice the methods of the invention are well described in the patent and scientific literature, and some illustrative examples are set forth below.

The GR antagonists used in the methods of the invention can be administered by any means known in the art, e.g., parenterally, topically, orally, or by local administration, such as by aerosol or transdermally. The methods of the invention provide for prophylactic and/or therapeutic treatments. The GR antagonists as pharmaceutical formulations can be administered in a variety of unit dosage forms depending-upon the condition, disease or disorder, the general medical-condition of each subject, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

GR antagonist pharmaceutical formulations can be prepared according to any method known to the art for the manufacture of pharmaceuticals. Such drugs can contain sweetening agents, flavoring agents, coloring agents and preserving agents. Any GR antagonist formulation can be admixtured with nontoxic pharmaceutically acceptable excipients which are suitable for manufacture.

Pharmaceutical formulations for oral administration can be formulated using pharmaceutically acceptable carriers well known in the art in appropriate and suitable dosages. Such carriers enable the pharmaceutical formulations to be formulated in unit dosage forms as tablets, pills, powder, dragees, capsules, liquids, lozenges, gels, syrups, slurries, suspensions, etc., suitable for ingestion by the subject. Pharmaceutical preparations for oral use can be obtained through combination of GR antagonist compounds with a solid excipient, optionally grinding a resulting mixture, and processing the mixture of granules, after adding suitable additional compounds, if desired, to obtain tablets or dragee cores. Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain GR antagonist mixed with a filler or binders such as lactose or starches, lubricants such as talc or magnesium stearate, and, optionally, stabilizers. In soft capsules, the GR antagonist compounds may be dissolved or suspended in suitable liquids, such as fatty oils, liquid paraffin, or liquid polyethylene glycol with or without stabilizers.

Aqueous suspensions of the invention contain a GR antagonist in admixture with excipients suitable for the manufacture of aqueous suspensions. Such excipients include a suspending agent, such as sodium carboxymethylcellu lose, methylcellu lose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending a GR antagonist in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

Dispersible powders and granules of the invention suitable for preparation of an aqueous suspension by the addition of water can be formulated from a GR antagonist in admixture with a dispersing, suspending and/or wetting agent, and one or more preservatives. Suitable dispersing or wetting agents and suspending agents are exemplified by those disclosed above. Additional excipients, for example, sweetening, flavoring and coloring agents, can also be present.

The GR antagonists of this invention can also be administered in the form of suppositories for rectal administration of the drug. These formulations can be prepared by mixing the drug with a suitable non-irritating excipient which is solid at ordinary temperatures but liquid at the rectal temperatures and will therefore melt in the rectum to release the drug. Such materials are cocoa butter and polyethylene glycols.

The GR antagonists of this invention can also be administered by in intranasal, intraocular, intravaginal, and intrarectal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111).

The GR antagonists of the invention can be delivered by transdermally, by a topical route, formulated as applicator sticks, solutions, suspensions, emulsions, gels, creams, ointments, pastes, jellies, paints, powders, and aerosols.

The GR antagonists of the invention can also be delivered as microspheres for slow release in the body. For example, microspheres can be administered via intradermal injection of drug (e.g., mifepristone)-containing microspheres, which slowly release subcutaneously (see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674). Both transdermal and intradermal routes afford constant delivery for weeks or months.

The GR antagonist pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the GR antagonist formulations of the invention are useful for parenteral administration, such as intravenous (IV) administration or administration into a body cavity or lumen of an organ. The formulations for administration will commonly comprise a solution of the GR antagonist (e.g., mifepristone) dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of GR antagonist in these formulations can vary widely, and will be selected primarily based on fluid volumes, viscosities, body weight, and the like, in accordance with the particular mode of administration selected and the subject's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the GR antagonist formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR antagonist into the target cells in vivo See, e.g., Al-Muhammed (1996) J. MicroencapsuL 13:293-306; Chonn (1995) Curr. Opin. Biotechnol. 6:698-708; Ostro (1989) Am. J. Hosp. Pharm. 46:1576-1587.

The amount of GR antagonist adequate to modulate the circadian rhythm of a subject is defined as a “therapeutically effective dose.” The dosage schedule and amounts effective for this use, i.e., the “dosing regimen,” will depend upon a variety of factors, including the stage of the disease or condition, the severity of the disorder or condition, the general state of the subject's health, the subject's physical status, age and the like. In calculating the dosage regimen for a subject, the mode of administration also is taken into consideration.

The dosage regimen also takes into consideration pharmacokinetics parameters well known in the art, i.e., the GR antagonists' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J Clin. Pharmacol. 24:103-108; the latest Remington's, supra). For example, in one study, less than 0.5% of the daily dose of mifepristone was excreted in the urine; the drug bound extensively to circulating albumin (see Kawai (1989) supra). The state of the art allows the clinician to determine the dosage regimen for each individual subject, GR antagonist and disease or condition treated. As an illustrative example, the guidelines provided below for mifepristone can be used as guidance to determine the dosage regiment, i.e., dose schedule and dosage levels, of any GR antagonist administered when practicing the methods of the invention.

Single or multiple administrations of GR antagonist formulations can be administered depending on the dosage and frequency as required and tolerated by the subject. The formulations should provide a sufficient quantity of active agent, e.g., mifepristone, to effectively modulate the circadian rhythm of a subject. Thus, one typical pharmaceutical formulation for oral administration of a GR antagonist, such as mifepristone, is administered in a daily amount of between about 2 to about 20 mg per kilogram of body weight per day, such as in a daily amount of between about 2 to about 12 mg per kilogram of body weight per day, including in a daily amount of between about 2 to about 9 mg per kilogram of body weight per day.

Lower dosages can be used, particularly when the drug is administered to an anatomically secluded site, in contrast to administration orally, into the blood stream, into a body cavity or into a lumen of an organ. Substantially higher dosages can be used in topical administration. Actual methods for preparing parenterally administrable GR antagonist formulations will be known or apparent to those skilled in the art and are described in more detail in such publications as Remington's, supra. See also Nieman, In “Receptor Mediated Antisteroid Action,” Agarwal, et al., eds., De Gruyter, New York (1987).

After a pharmaceutical comprising a GR antagonist of the invention has been formulated in an acceptable carrier, it can be placed in an appropriate container and labeled for use in the subject methods. For administration of GR antagonist, such labeling would include, e.g., instructions concerning the amount, frequency and method of administration.

As summarized above, in some embodiments, the subject method of modulating the circadian rhythm of a subject is achieved by administering to the subject an effective amount of a GR antagonist and an effective amount of an additional compound, such as a corticotropin releasing hormone (CRH) antagonist or a mineralcorticoid receptor (MR) agonist. In other embodiments the subject method of modulating the circadian rhythm of a subject is achieved by administering to the subject an effective amount of a CRH antagonist. In yet other embodiments, subject method of modulating the circadian rhythm of a subject is achieved by administering to the subject an effective amount of a MR agonist.

CRH antagonists suitable for use with the subject invention include, but are not limited to the following, antalarmnin, R-121919, astressin, and the like.

MR agonist suitable for use with the subject invention include, but are not limited to the following, deoxycorticosterone, spironolactone, fludrocortisone, aldosterone, progesterone, and the like.

In the above embodiments where CRH antagonists and/or MR agonists are employed, these agents can be administered using any convenient formulation and protocol, such as the protocols described above in connection with the administration of GR antagonists.

Modulating of the circadian rhythm of a subject can be readily evaluated by methods apparent to one skilled in the art. Such methods of evaluating whether the circadian rhythm of a subject has been modulated include determining whether the subject exhibits an increased capability of controlling initiation of the sleep stage of the sleep cycle, or controlling the natural termination of the sleep stage of the sleep cycle or physiologically adapting to an environmentally imposed change in sleep cycle. Control of initiation of the sleep stage means the subject is capable of initiating the sleep stage of the sleep cycle at a particular desired time point. As such, control of initiation of the sleep stage includes the ability by the subject to initiate the sleep stage of the sleep cycle at a particular time, where such time is earlier than the usual expected time, i.e., occurring before the usual expected time, where such time is at least about 30 minutes to about 12 hours, such as at least about 2 to about 10 hours, including at least about 3 to about 7 hours before the usual expected time. Control of initiation of the sleep stage also includes the ability by the subject to initiate the sleep stage of the sleep cycle at a particular time, where such time is later than the usual expected time, i.e., occurring after the usual expected time, where such time is at least about 30 minutes to about 12 hours, such as at least about 2 to about 10 hours, including at least about 3 to about 7 hours after the usual expected time.

Control of the natural termination of the sleep stage means the subject is capable of naturally terminating the sleep stage of the sleep cycle at a particular desired time point. As such, control of initiation of the sleep stage includes the ability by the subject to wake up from the sleep stage of the sleep cycle at a particular time, where such time is earlier than the usual expected time, i.e., occurring before the usual expected time, where such time is at least about 30 minutes to about 12 hours, such as at least about 2 to about 10 hours, including at least about 3 to about 7 hours before the usual expected time. Control of natural termination of the sleep stage also includes the ability by the subject to wake up from the sleep stage of the sleep cycle at a particular time, where such time is later than the usual expected time, i.e., occurring after the usual expected time, where such time is at least about 30 minutes to about 12 hours, such as at least about 2 to about 10 hours, including at least about 3 to about 7 hours after the usual expected time.

Control of physiologically adapting to an environmentally imposed change in sleep cycle means the subject is required to sleep during a new time schedule and that one or more physiological circadian rhythms (i.e. temperature rhythm, cortisol rhythm, melatonin rhythm, ACTH rhythm, etc.) is phase advanced or phase delayed in a desired direction to help adapt to the imposed change in sleep cycle. As such, control of modifying one or more physiological circadian rhythms is determined by measuring a predetermined phase marker, where such time is earlier than the usual expected time, i.e., occurring before the usual expected time, where such time is at least about 30 minutes to about 12 hours, such as at least about 2 to about 10 hours, including at least about 3 to about 7 hours before the usual expected time. As such, control of modifying one or more physiological circadian rhythms is determined by measuring a predetermined phase marker, where such time is earlier than the usual expected time, i.e., occurring before the usual expected time, where such time is at least about 30 minutes to about 12 hours, such as at least about 2 to about 10 hours, including at least about 3 to about 7 hours before the usual expected time, where such time is at least about 30 minutes to about 12 hours, such as at least about 2 to about 10 hours, including at least about 3 to about 7 hours after the usual expected time.

In some embodiments, where subjects are unable to initiate sleep or have difficulty in initiating sleep, and where a phase advancement of the sleep cycle is desired, modulation of the circadian rhythm of a subject can be determined by evaluating the subject's ability to initiate sleep at a particular desired time. In such embodiments, an increase in the subject's ability to initiate sleep at a particular desired time means, for example, the following: an increase in the value which is calculated from the time that a subject sleeps divided by the time that a subject is attempting to sleep (e.g., total time that a subject maintains sleep divided by the total time the subject is attempting to initiate sleep), where such an increase is at least about 1.2 or more fold, such as at least about 3 or more fold, including at least about 4, 6, 8 fold or even higher, compared to a control; an increase in the total amount of sleep, where such an increase is at least about 1.2 or more fold, such as at least about 3 or more fold, including at least about 4, 6, 8 fold or even higher, compared to a control; a decrease in sleep latency (the time it takes to fall asleep), where such a decrease is at least about 1.2 or more fold, such as at least about 3 or more fold, including at least about 4, 6, 8 fold or even higher, compared to a control; and a decrease in the time spent awake following the initial attempt to initiate a sleep stage, where such a decrease is at least about 1.2 or more fold, such as at least about 3 or more fold, including at least about 4, 6, 8 fold or even higher, compared to a control.

Other methods of evaluating modulation of the circadian rhythm of a subject include any testing methods involving interrogating the subject and determining the effectiveness of the administration of the GR antagonist on modulating the subject's circadian rhythm. Such evaluation may include first determining when the subject-desires to initiate the sleep stage of the sleep cycle prior to administration of the GR antagonist, and then determining when the subject was actually able to initiate the sleep stage of the sleep cycle after administration of the GR antagonist.

Utility

The subject methods may be used in treating a subject suffering from a sleep disorder, including for example, sleep problems associated with insomnia, Circadian Rhythm Sleep Disorder, Delayed Sleep Phase Type, Circadian Rhythm Sleep Disorder, Advanced Sleep Phase Type, shift-work induced sleep disorder, Circadian Rhythm Sleep Disorder, Jet Lag Type, Circadian Rhythm Sleep Disorder, Irregular Sleep-Wake Type, Circadian Rhythm Sleep Disorder, Free-Running Type, short sleeper, long sleeper and the like. The methods may also be useful for treating HPA axis abnormalities in subjects suffering from a sleep-related breathing disorder, such as obstructive sleep apnea, wherein such HPA axis abnormalities may contribute to insomnia. As such, the subject methods are useful for treating subjects that are unable to initiate sleep and maintain sleep, or unable to initiate sleep or maintain sleep, such as subjects suffering from insomnia, have difficulty in initiating sleep, such as subjects suffering from Circadian Rhythm Sleep Disorder, Delayed Sleep Phase Type, or are subjects having difficulty in maintaining an awake state prior to a desired time of sleep initiation, such as subjects suffering from Circadian Rhythm Sleep Disorder, Advanced Sleep Phase Type.

The present invention is further useful for subjects desiring an adaptation of the circadian rhythm to a different time schedule. For example, air travelers who rapidly cross two or more time zones may find their internal circadian clocks out of phase with the day/night cycle at their destination, giving rise to the so-called “jet-lag” syndrome in which they suffer disruptions of their sleep patterns and diminished attention span and alertness until their inner biological clocks gradually adjust to local time, will find use in modulating their circadian rhythm in order to phase shift their circadian rhythm to adapt to the environmentally imposed desired sleep cycle In addition subjects, whose work schedules rotate among day shift, night shift and the so-called “graveyard” shift, and experience transient internal temporal dissociation or a lack of synchronization among various bodily rhythms, and consequent difficulty in adjusting to shift changes, will also find use in modulating their circadian rhythm in order to phase shift their circadian rhythm to adapt the environmentally imposed desired sleep cycle.

Thus, in one aspect the subject methods provides for treating a subject, where the subject suffers from a sleep disorder. As used herein the term “sleep disorder” refers to a disordered, interrupted or fragmented sleep characterized by events including, but not limited to, snoring, periods of sleep apnea, narcolepsy, restless legs syndrome, sleep terrors, sleep walking, and daytime somnolence. Such sleep disorders include, but are not limited to, insomnia, Circadian Rhythm Sleep Disorder, Delayed Sleep Phase Type, Circadian Rhythm Sleep Disorder, Advanced Sleep Phase Type, shift-work induced sleep disorder, Circadian Rhythm Sleep Disorder, Jet Lag Type, Circadian Rhythm Sleep Disorder, Irregular Sleep-Wake Type, Circadian Rhythm Sleep Disorder, Free-Running Type, short sleeper, long sleeper and HPA axis abnormalities associated with sleep apnea that may interfere with sleep or contribute to metabolic complications of sleep apnea and the like.

In another aspect the subject method provides for treating a subject, where the subject desires an adaptation to a differing time schedule, such as for example, inter-time zone travel induced sleep disturbance (Circadian Rhythm Sleep Disorder, Jet Lag Type) (i.e., jet-lag), shift workers' sleep disturbances, and the like. Sleep disorders and sleep disturbances are generally characterized by difficulty in initiating or maintaining sleep or in obtaining restful or enough sleep.

Treatment of a subject according to the subject methods can be correlated to an enhancement of sleep quality of the subject. An enhancement of sleep quality may be determined, for example, by examining the following parameters in a subject after administration of the glucocorticoid receptor antagonist: an increase in the value which is calculated from the time that a subject sleeps divided by the time that a subject is attempting to sleep; a decrease in sleep latency (the time it takes to fall asleep); a decrease in the number of awakenings during sleep stage; a decrease in the time spent awake following the initial attempt to initiate a sleep stage; an increase in the total amount of sleep; an increase the amount and percentage of REM sleep; an increase in the duration and occurrence of REM sleep; a decrease in the fragmentation of REM sleep; an increase in the amount and percentage of slow-wave (i.e. stage 3 or 4) sleep; an increase in the amount and percentage of stage 2 sleep; a decrease in the number of awakenings, especially in the early morning; an increase in daytime alertness; and increased sleep maintenance; a change in the distribution of REM and SWS during the night (e.g., a change in the amount of SWS in the first half of the night, or a change in the amount of REM in the second half of the night). Wherein an increase in parameters noted above means an increase of at least about 1.2 or more fold, such as at least about 3 or more fold, including at least about 4, 6, 8 fold or even higher, compared to a control, and wherein a decrease in the parameters noted above means a decrease of at least about 1.2 or more fold, such as at least about 3 or more fold, including at least about 4, 6, 8 fold or even higher, compared to a control;

Thus, in one aspect the subject methods provide for treating a subject for a sleep disorder by administering to the subject an effective amount of a glucocorticoid receptor antagonist in a manner sufficient to modulate the circadian rhythm of the subject. In some embodiments the subject has already been identified as suffering form a sleep disorder. In other embodiments, the subject is first identified as suffering from a sleep disorder.

A sleep disorder is characterized by a difficulty in the ability of the subject to initiate or maintain sleep or difficulty in obtaining restful or enough sleep. Examples of sleep disorders include, but are not limited to, insomnia, Circadian Rhythm Sleep Disorder, Advanced Sleep Phase Type, Circadian Rhythm Sleep Disorder, Delayed Sleep Phase Type, shift-work induced sleep disorder, Circadian Rhythm Sleep Disorder, Jet Lag Type, Circadian Rhythm Sleep Disorder, Irregular Sleep-Wake Type, Circadian Rhythm Sleep Disorder, Free-Running Type, short sleeper, long sleeper and HPA axis abnormalities associated with sleep apnea that may interfere with sleep and the like.

Insomnia can be classified as transient, occurring only for a short term and, intermittent, occurring from time to time, and chronic, occurring on most nights and lasts a month or more. Symptoms of insomnia can be different for each individual, and subjects suffering form insomnia might experience a variety of symptoms, such as: difficulty falling asleep, which can mean lying in bed for up to an hour or more, including tossing and turning; awakening during sleep and having trouble getting back to sleep; awakening too early in the morning; feeling unrefreshed upon awakening; and daytime irritability, drowsiness, anxiety, and/or nonproductiveness.

Circadian Rhythm Sleep Disorder, Advanced Sleep Phase Type is a sleep disorder in which the sleep cycle is advanced in relation to the desired clock time, resulting in symptoms of compelling evening sleepiness, an early sleep onset, and an awakening that is earlier than desired. Symptoms of Circadian Rhythm Sleep Disorder, Advanced Sleep Phase Type include, for example, the following: inability to stay awake until the desired bedtime or inability to remain asleep until the desired time of awakening; a phase advance of the sleep cycle in relation to the desired time for sleep; and symptoms last for at least about 2 to 4 months, including at least about 3 months. When not required to remain awake until the later bedtime, subjects suffering from Circadian Rhythm Sleep Disorder, Advanced Sleep Phase Type will generally have a habitual sleep period that is of normal quality and duration, with a sleep onset earlier than desired, awaken spontaneously earlier than desired, and maintain stable entrainment to a 24-hour sleep-wake pattern.

Circadian Rhythm Sleep Disorder, Delayed Sleep Phase Type is a sleep disorder in which the sleep cycle is delayed by at least about 1.5 or more hours, including at least about 2 or more hours of the desired bedtime. A subject suffering from such a sleep disorder will generally have difficulty awakening at the desired time. Symptoms of Circadian Rhythm Sleep Disorder, Delayed Sleep Phase Type include, for example, the following: complaint of insomnia or excessive sleepiness; inability to fall asleep at the desired time; inability to wake up at the desired time; depression may be present; and such a sleep pattern lasts for at least about 2 to 4 months, including at least about 3 months.

Short sleeper syndrome is characterized as a an individual who habitually and spontaneously sleeps substantially less in a 24-hour period than is expected for a person in his or her age group, and does not have excessive sleepiness. Individuals that have short sleeper syndrome awaken spontaneously and have a daily total sleep time of less than about 65% to about 90%, such as about 70% to about 80%, including about 75% of the age-related norm.

Long sleeper syndrome is characterized as an individual who habitually and spontaneously sleeps substantially more in a 24-hour period than is expected for a person in his or her age group. Individuals that have long sleeper syndrome have a daily total sleep time of more than about 1 hour to about 4 hours, such as about 2 hours to about 3 hours, including about 2.5 hours of the age-related norm.

Obstructive sleep apnea is characterized as a cessation of breathing during sleep that is caused by repetitive partial or complete obstruction of the airway by pharyngeal structures. Patients with obstructive sleep apnea often are overweight, snore loudly, and complain of daytime fatigue and sleepiness. The severity of the condition is measured by the number of apneas (i.e., cessations of airflow) or hypopneas (i.e., reductions in airflow) that cause sleep arousal.

Upper airway resistance syndrome characterized by a partial collapse of the upper airway resulting in increased resistance to airflow. In contrast to Obstructive Sleep Apnea it is seen more often in women than men and snoring is the hallmark clinical symptom. Multiple sleep fragmentations are measured by short alpha EEG arousals but the syndrome does not result in apneic and hypopneic events.

In such embodiments, the subject is administered an effective amount of a glucocorticoid receptor antagonist in order to modulate the subject's circadian rhythm, either by changing the phase or changing the shape of the waveform, thereby altering the subject's sleep.

Subjects suffering from a sleep disorder characterized by an inability to initiate and/or maintain sleep, such as insomnia, may be treated according to the subject methods by administering to the subjects an effective amount of a glucocorticoid receptor antagonist in order to modulate the subjects' circadian rhythm waveform. In such embodiments, in which modifying the subject's circadian rhythm waveform is desired, a glucocorticoid receptor antagonist may be administered to the subjects in a daily amount of between about 2 to about 20 mg per kilogram of body weight per day, such as in a daily amount of between about 2 to about 12 mg per kilogram of body weight per day, including in a daily amount of between about 8 to about 12 mg per kilogram of body weight per day. The dose may be given in the morning. The dose may be given on either an intermittent or chronic basis.

Subjects suffering from a sleep disorder characterized by delayed ability to initiate sleep, such as Circadian Rhythm Sleep Disorder, Delayed Sleep Phase Type, may be treated according to the subject methods by administering to the subjects an effective amount of a glucocorticoid receptor antagonist in order to modulate the subjects' circadian rhythm and phase advancing the subjects' sleep cycle. In such embodiments, in which phase advancing the subjects' sleep cycle is desired, a glucocorticoid receptor antagonist may be administered to the subjects in a daily amount of between about 2 to about 20 mg per kilogram of body weight per day, such as in a daily amount of between about 2 to about 12 mg per kilogram of body weight per day, including in a daily amount of between about 5 to about 9 mg per kilogram of body weight per day. The dose may be given in at a time of day designed to advance the subject's circadian rhythm. The dose may be given on either an intermittent or chronic basis.

Subjects suffering from a sleep disorder characterized by an inability to maintain an awake state prior to desired time of sleep initiation, such as Circadian Rhythm Sleep Disorder, Advanced Sleep Phase Type, may be treated according to the subject methods by administering to the subjects an effective amount of a glucocorticoid receptor antagonist in order to modulate the subjects' circadian rhythm and phase delay the subjects' sleep cycle. In such embodiments, which phase delaying the subjects' sleep cycle is desired, a glucocorticoid receptor antagonist may be administered to the subject in a daily amount of between about 2 to about 20 mg per kilogram of body weight per day, such as in a daily amount of between about 2 to about 12 mg per kilogram of body weight per day, including in a daily amount of between about 5 to about 9 mg per kilogram of body weight per day. The dose may be given at a time of day designed to delay the subject's circadian rhythm. The dose may be given on either an intermittent or chronic basis.

In another aspect, the subject methods provide for treating a subject desiring an adaptation of the subject's circadian rhythm to a different time schedule. In some embodiments, the subject methods may be used to treat air travelers who rapidly cross two or more time zones, such as three to five time zones. In such embodiments, the subjects may find their internal circadian clocks out of phase with the day/night cycle at their destination, giving rise to the so-called “jet-lag” syndrome, thereby suffering disruptions of their sleep patterns and diminished attention span and alertness until the inner biological clocks of the subjects' gradually adjust to local time. The subject methods may be suitable for treating subjects experiencing an inter-time zone travel induced sleep disturbance (Circadian Rhythm Sleep Disorder, Jet Lag Type) desiring a phase advancement of the sleep cycle or a phase delay or the sleep cycle. In such embodiments, a glucocorticoid receptor antagonist may be administered to a subject in a daily amount of between about 2 to about 20 mg per kilogram of body weight per day, such as in a daily amount of between about 2 to about 12 mg per kilogram of body weight per day, including in a daily amount of between about 5 to about 9 mg per kilogram of body weight per day. The dose may be given in at a time of day designed to achieve a desirable advance or delay the subject's circadian rhythm, to thereby help synchronize the subject to the new time zone. The dose may be given on either an intermittent basis.

In another embodiment, the subject method can be used to treat subjects whose work schedules rotate among day shift, night shift and the so-called “graveyard” shift, and experience transient internal temporal dissociation or a lack of synchronization among various bodily rhythms, and consequently experience difficulty in adjusting to shift changes. The subject methods may be suitable for treating a subject experiencing a sleep disturbance resulting from shift work, where the subject desires a phase advancement of the sleep cycle or a phase delay or the sleep cycle. In such embodiments, the subject may be administered a glucocorticoid receptor antagonist in a daily amount of between about 2 to about 20 mg per kilogram of body weight per day, such as in a daily amount of between about 2 to about 12 mg per kilogram of body weight per day, including in a daily amount of between about 5 to about 9 mg per kilogram of body weight per day. The dose may be given in at a time of day designed to achieve a desirable advance or delay the subject's circadian rhythm, to thereby help synchronize the subject to their desired schedule. The dose may be given on either an intermittent or chronic basis.

Kits

Also provided by the subject invention are kits for use in the subject methods described above. Kits for practicing the subject methods include a glucocorticoid receptor antagonist. In some embodiments, the glucocorticoid receptor antagonist includes a steroidal skeleton with at least one phenyl-containing moiety in the 11-beta position of the steroidal skeleton. In further embodiments, the phenyl-containing moiety in the 11-beta position of the steroidal skeleton is a dimethylaminophenyl moiety. In yet further embodiments, the glucocorticoid receptor antagonist is mifepristone (i.e., RU486), RU009, RU044, ORG-34517, ORG-34850, or ORG-34116.

The kits may further include means for delivering the glucocorticoid receptor antagonist to the subject, e.g. a syringe. The subject kits further typically include instructions for carrying out the subject methods, where these instructions may be present on a package insert and/or the packaging of the kit. Such instructions may include instructions for using the glucocorticoid receptor antagonist for treating a subject suffering from a sleep disorder such as insomnia, Circadian Rhythm Sleep Disorder, Delayed Sleep Phase Type, and Circadian Rhythm Sleep Disorder, Advanced Sleep Phase Type, or phase shifting a host's sleep cycle for treating a sleep disturbance resulting from inter-time zone travel (i.e., jet-lag) or shift-work. Such instruction may further include information such as dosage and schedule of administration of the GR antagonist. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE 1

Mifepristone for Treatment of Insomnia: Polysomnogram Measures

Recently, HPA axis hyperactivity has been reported in some forms of insomnia (Rodenbeck and Hajak, J Clin Psychiatry. 2001 June; 62(6):453-63; Vgontzas et al., J Clin Endocrinol Metab. 2001 August; 86(8):3787-94), without depression. A resetting of the HPA axis with a GR/PR antagonist has been described in psychiatric patients (Belanoff et al., Biol. Psych. Sep. 1, 2002; 52(5):386-92 ). An early report of mifepristone in a healthy male reported significant worsening of sleep with mifepristone (Wiedemann et al., Eur Arch Psychiatry Clin Neurosci. 1992; 241(6):372-5). In this latter study, mifepristone was given in the afternoon and its longitudinal effect with time, after discontinuation was not measured.

In this double-blinded placebo controlled study the longitudinal effects of 5 days of a GR antagonist on HPA axis activity, melatonin and sleep in subjects with insomnia were measured. A 30 day repeated study incorporating HPA axis activity (e.g., cortisol and ACTH) as well as melatonin and sleep changes before, during, and after discontinuation of treatment were measured. The design was formulated to additionally establish the temporal relationship between treatment and response, including determination of measurements two weeks after discontinuation of treatment. During the study all the subjects were put to bed at usual bed-time. The subjects were administered 600 mg of mifepristone per day in the morning for five consecutive days. All subjects were permitted to sleep up until natural wake-up, limited to 7:45 to 8:00 am to allow for evaluation of increased natural total sleep time.

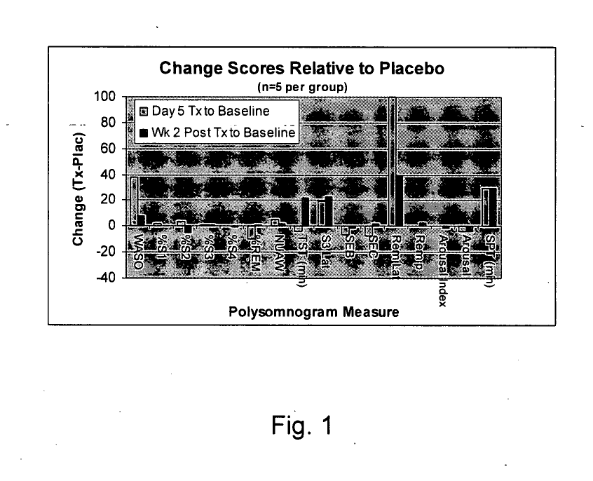

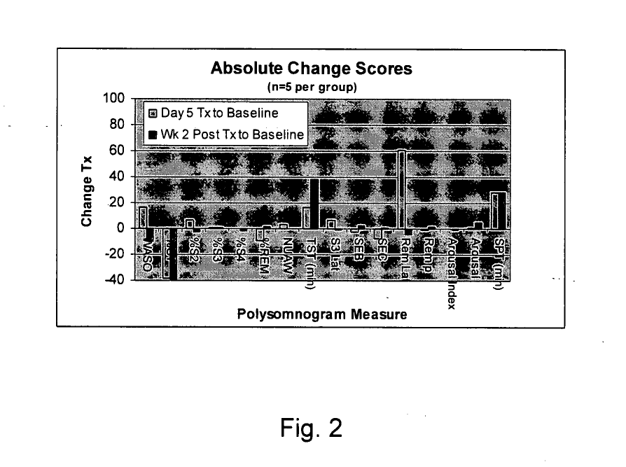

The results show that as a result of the administration of mifepristone, total sleep time (TST) increased (at 2 weeks post treatment) after treatment (FIGS. 1 and 2, Tables 1 and 2). This result was seen in PSG as well as during outpatient portion by prospective actigraphy measures. In addition, REM latency (RL) increased (most acutely during active treatment), which is important with respect to the beneficial effect of mifepristone administration in depression (FIGS. 1 and 2, Tables 1 and 2) and in narcolepsy. Since REM latency changes with the phase of the circadian core temperature rhythm, this shows that the circadian rhythm of temperature is delayed with treatment as well.

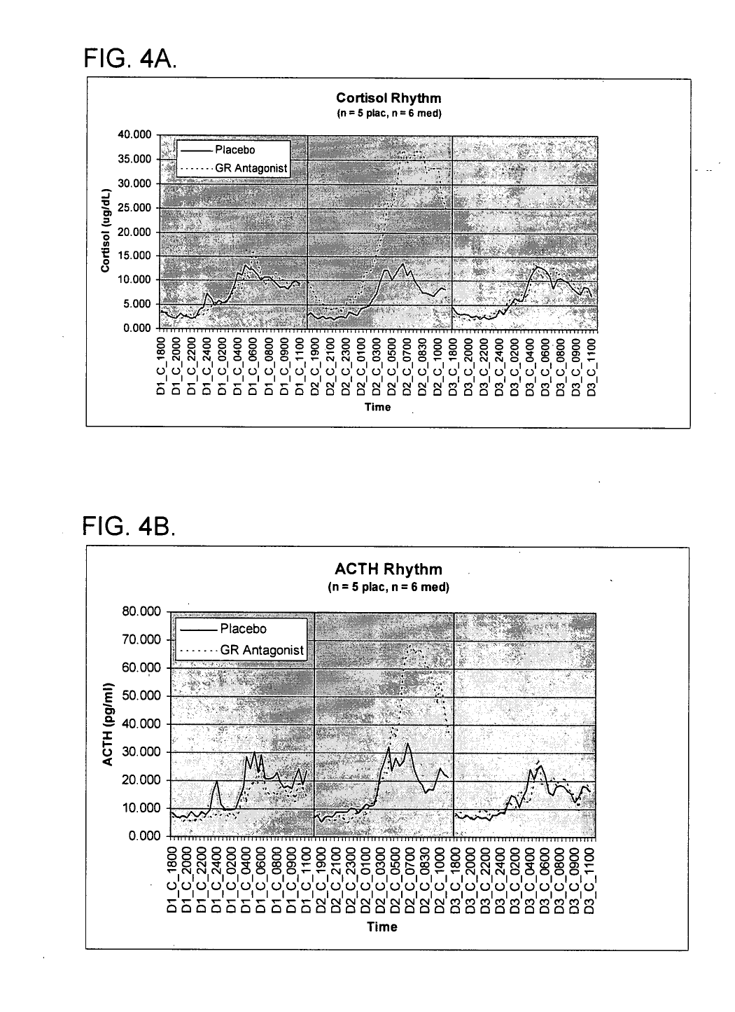

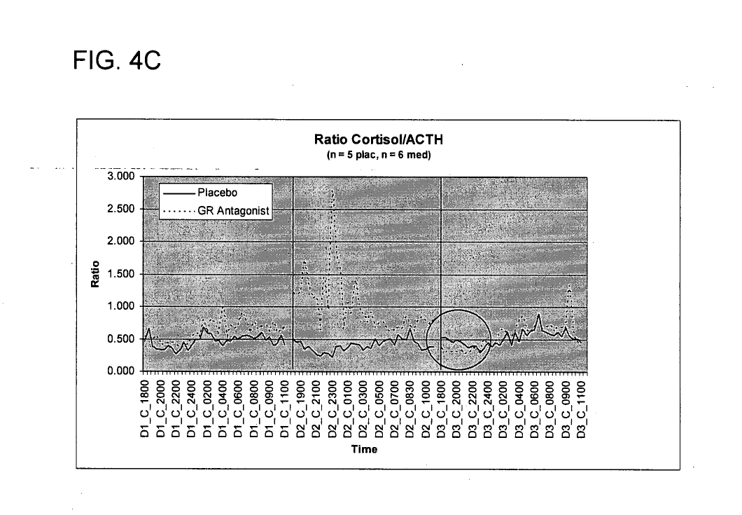

Sleep changes are associated with changes in ACTH, cortisol and melatonin (acutely) and after treatment cessation. FIG. 4 shows increased in the ratio of ACTH to cortisol post-treatment. One possibility is that this may represent decreased central CRH after treatment discontinuation with subsequent decreased direct brainstem norepinephrine activation by brain CRH.

TABLE 1
Change in Polysomnogram Scores Treatment Group
(Data set includes 5 double blinded treatment patients,
and 5 double blinded placebo patients)

| | | | | 2 Wk | | |
|---|---|---|---|---|---|---|
| | Day 5 Tx | | | Post Tx | | |
| | Rel to | | | Rel to | | |
| | Baseline | | Tx | Baseline | | Tx |
| | Tx | Plac | rel to | Tx | Plac | rel to |
| | Delta | Delta | Plac | Delta | Delta | Plac |
| WASO | 16.4 | −21.45 | 37.85 | −10.4 | −18.25 | 7.85 |
| (min) | | | | | | |
| % S1 | −38.34 | −40.1 | 1.76 | −39.19 | −39.18 | −0.01 |
| % S2 | 6.44 | 2.54 | 3.9 | −2.68 | 2.37 | −5.05 |
| % S3 | 0.76 | 0.05 | 0.71 | 1.09 | −0.36 | 1.45 |
| % S4 | −0.01 | −0.43 | 0.42 | −1.48 | −0.5 | −0.98 |
| % REM | −9.33 | 0.36 | −9.69 | 1.63 | 0.32 | 1.31 |
| NUAW | 3.3 | −1.7 | 5 | 0 | −1.5 | 1.5 |
| TST (min) | 17.05 | 20.8 | −3.75 | 38.5 | 16.85 | 21.65 |
| S3 Lat | 7.05 | −12.3 | 19.35 | −0.85 | −23.95 | 23.1 |
| (min) | | | | | | |
| SEB | −2.13 | 4.72 | −6.85 | 2.57 | 3.91 | −1.34 |
| (S2, S3, | | | | | | |
| S4) | | | | | | |
| SEC (S3, | −7.3 | 0.15 | −7.45 | 1.19 | −0.83 | 2.02 |
| S4) | | | | | | |
| Rem Lat | 61.15 | −38.45 | 99.6 | −4 | −43.5 | 39.5 |
| (min) | | | | | | |
| Rem p | −0.826 | −1.48 | 0.654 | 1.972 | −0.758 | 2.73 |
| Arousal | −0.35 | −0.64 | 0.29 | −0.85 | 0.24 | −1.09 |
| Index | | | | | | |
| Arousal | 0.9 | 4.7 | −3.8 | 5.2 | 8.9 | −3.7 |
| SPT (min) | 28.69 | −0.7 | 29.39 | 28.1 | −1.45 | 29.55 |

Definitions:
WASO = wake after sleep onset
% S1 = percentage stage 1 sleep
% S2 = percentage stage 2 sleep
% S3 = percentage stage 3 sleep
% S4 = percentage stage 4 sleep
% REM = percentage REM sleep
NUAW = number of awakenings
TST = total combined minutes of sleep
S3 Lat (min) = latency to onset of stage 3 sleep
SEB = total combined minutes of stage 2, 3 and 4 per total sleep minutes
SEC = total combined minutes of stage 3, 4 per total sleep minutes
% NEM = sum of % S3 and % S4
Rem lat = minutes to first epoch of REM after sleep onset
REM density = # rapid eye movements per minutes of REM sleep
Arousal index = computerized measure high frequency bands/minutes sleep

TABLE 2
Average Polysomnogram Measures (Data set includes 5 double blinded
treatment patients, and 5 double blinded placebo patients)

| | Rx | | Placebo | | Rx | | Placebo | | Rx | | Placebo | |
|---|---|---|---|---|---|---|---|---|---|---|---|---|
| | Ave | | Ave | | Ave | | Ave | | Ave | | Ave | |
| | Rx | T1 | Plac | | Rx | T2 | Plac | | Rx | T3 | Plac | |
| | (n = 5) | SD | (n = 5) | SD | (n = 5) | SD | (n = 5) | SD | (n = 5) | SD | (n = 5) | SD |
| WASO (min) | 66.95 | 24.22 | 83.35 | 69.71 | 83.35 | 42.34 | 61.90 | 37.43 | 56.55 | 19.73 | 65.10 | 44.75 |
| % S1 | 13.21 | 6.62 | 11.70 | 3.97 | 15.48 | 9.65 | 9.10 | 2.72 | 14.63 | 6.40 | 10.02 | 2.07 |
| % S2 | 53.82 | 7.63 | 49.20 | 8.82 | 60.26 | 11.60 | 51.74 | 6.03 | 51.14 | 1.96 | 51.57 | 6.17 |
| % S3 | 6.87 | 4.57 | 10.92 | 5.00 | 7.63 | 5.87 | 10.97 | 5.30 | 7.96 | 4.32 | 10.56 | 5.39 |
| % S4 | 4.37 | 8.25 | 6.22 | 5.58 | 4.36 | 8.37 | 5.79 | 5.49 | 2.89 | 4.77 | 5.72 | 4.61 |
| % REM | 20.95 | 2.97 | 21.33 | 5.41 | 11.62 | 5.86 | 21.69 | 3.72 | 22.58 | 3.98 | 21.65 | 4.08 |
| NUAW | 24.30 | 10.47 | 25.30 | 10.90 | 27.60 | 10.39 | 23.60 | 18.46 | 24.30 | 15.53 | 23.80 | 16.37 |
| TST (min) | 373.60 | 74.21 | 364.20 | 64.40 | 390.65 | 61.95 | 385.00 | 21.34 | 412.10 | 58.34 | 381.05 | 26.64 |
| S3 Lat (min) | 17.75 | 14.49 | 39.45 | 40.38 | 24.80 | 23.44 | 27.15 | 19.63 | 16.90 | 10.39 | 15.50 | 3.62 |
| SE (S2, S3, S4) | 85.47 | 5.25 | 81.88 | 13.52 | 83.34 | 7.83 | 86.60 | 7.15 | 88.04 | 3.47 | 85.79 | 9.24 |
| SE (S3, S4) | 32.24 | 10.73 | 38.46 | 8.56 | 24.94 | 17.20 | 38.61 | 6.50 | 33.43 | 6.71 | 37.63 | 7.26 |
| Rem Lat (min) | 76.80 | 13.41 | 113.60 | 53.98 | 137.95 | 55.79 | 75.15 | 35.40 | 72.80 | 21.69 | 70.10 | 28.48 |
| Rem density | 7.99 | 3.16 | 9.68 | 5.14 | 7.16 | 3.73 | 8.20 | 4.54 | 9.95 | 2.99 | 8.92 | 4.60 |
| Arousal Index | 16.60 | 5.31 | 17.28 | 5.98 | 16.25 | 3.51 | 16.64 | 7.68 | 15.75 | 3.51 | 17.52 | 7.04 |
| Arousal | 104.80 | 39.64 | 101.30 | 27.06 | 105.70 | 30.24 | 106.00 | 50.67 | 110.00 | 36.40 | 110.20 | 47.17 |
| SPT (min) | 440.55 | 75.72 | 447.60 | 58.59 | 469.24 | 66.18 | 446.90 | 34.85 | 468.65 | 65.41 | 446.15 | 40.17 |

TABLE 3
Actigraphy Measures of TST Minutes and WASO

| | Placebo | Tx |
|---|---|---|
| | (n = 3) | (n = 2) |
| D7AV-smin | 485.76 | 458.86 |
| D7AV-waso | 39.19 | 45.14 |
| D20AV-smin | 452.33 | 448.40 |
| D20AV-waso | 28.13 | 51.70 |
| D26AV-smin | 468.33 | 473.08 |
| D26AV-waso | 32.22 | 31.67 |

### EXAMPLE 2

Mifepristone for Circadian Phase Shifting: Hormonal Measures

Further applications for administration of a glucocorticoid antagonist include shifting the circadian rhythm of healthy subjects in preparing for or rapidly adjusting internal hormonal rhythms during inter-time zone travel (e.g., travel between eastern United States to western United States) and for adapting to shift-work. For example, beneficial effects include an acute phase delay in melatonin rhythm (as measured by DLMO), acute phase delay in cortisol and ACTH acrophase, and delay in REM latency, as well as a delay in temperature rhythm, since REM latency is a marker for the phase of the temperature rhythm). This could be helpful in delaying one's internal circadian rhythms to better adapt to sleeping in later time zone (as would occur on traveling from East to West) and thus help ameliorate the symptoms of jet lag,

In the study described in Example 1, melatonin was additionally measured under dim lights to measure changes in-its circadian rhythm with time. Melatonin is considered to be a somewhat pure marker for the timing of the phase of the body clock, or supra-chiasmatic nucleus (SCN).

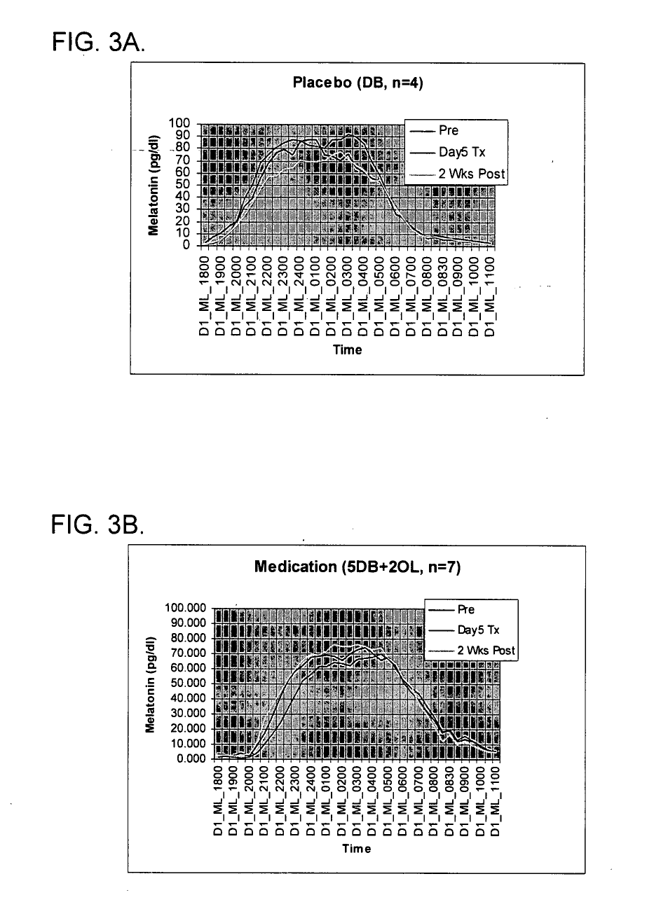

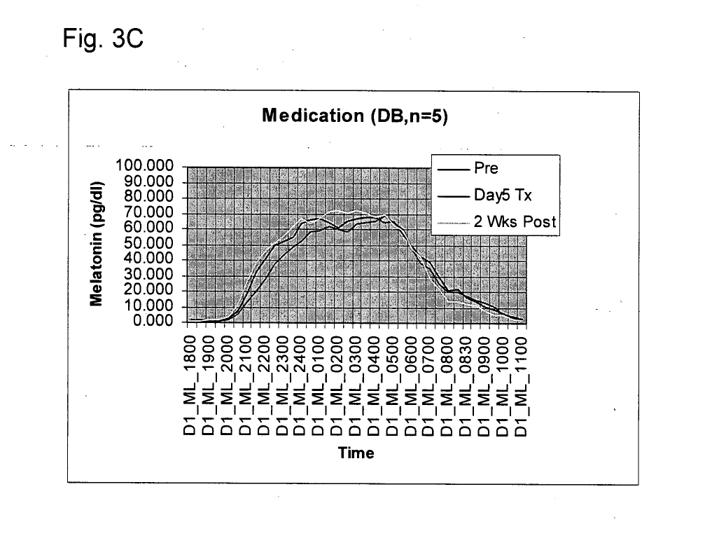

FIG. 3 shows absolute values of the timing of melatonin in both the treatment and placebo groups before and after 5 days of a GR antagonist as well as 2 weeks post-discontinuation These data shows a delay in DLMO (time at which melatonin reaches 20 pg/ml) in the GR antagonist group after 5 days treatment in the outpatient setting (which is consistent with a delay in the timing of the SCN).

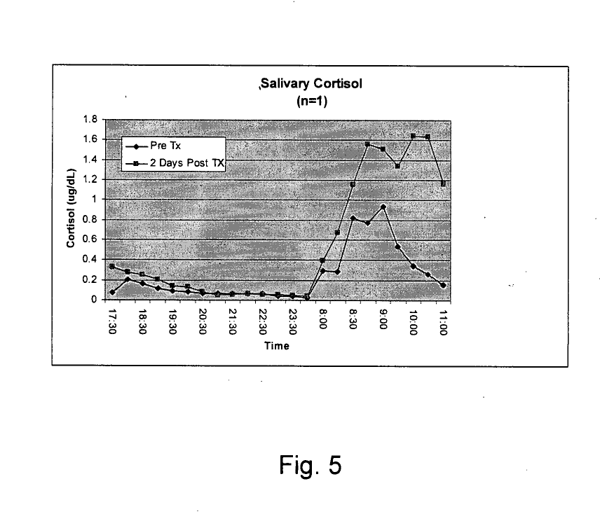

In a separate experiment, the longitudinal effects of 2 days of a GR antagonist on melatonin and HPA axis activity was measured in healthy control subject during an inpatient stay under constant routine conditions. In this repeated measure study design, a subject was given 400 mg of mifepristone for two consecutive days as a test protocol. Salivary cortisol, ACTH and dim light melatonin were measured the evening/morning before and on the second evening/morning of medication. The design was created to include measures of the phase of night-time DLMO as well as measures of the morning response of the HPA axis, including timing of cortisol acrophase. The design was formulated to ascertain circadian phase shifting in a controlled setting with healthy subjects (as might occur in jet-lag or shift work). FIG. 5 shows the results from a single subject during a test protocol. In addition to the expected rise in cortisol, this study shows a delay in the timing of the morning acrophase of cortisol with treatment. Salivary melatonin samples were insufficient.

### EXAMPLE 3

The Acute Effects of a Mineralocorticoid (MR) Agonist on Nocturnal Hypothalamic-Adrenal-Pituitary (HPA) Axis Activity in Healthy Controls

I. Methods:

A. Subjects

Nine healthy subjects were recruited from the community via flyers and internet postings. Subjects responding to ads were phone screened to determine if subjects met initial eligibility criteria. Those subjects meeting criteria underwent physical examination, screening labs (complete blood count, comprehensive metabolic panel, urine analysis, urine toxicology screen, TSH, FT4, serum pregnancy test) and EKG. A SCID was performed to rule out Axis I and Axis II DSM-TR pathology. At the time of this analysis and manuscript preparation, 9 subjects have been enrolled. Inclusion criteria were: (1) participants are age 18 to 85 years old with a HAM-D score less than or equal to 5. Exclusion criteria were: (1) personal history of Axis I or Axis II disorders; (2) active medical problems; (3) abuse of drugs or alcohol in the 6 months prior to study; (4) use of additional prescription medications, street drugs or alcohol during the week before the study; (5) currently pregnant or lactating.

B. Study Design

This study is a two day repeated measures study of fludrocortisone's acute effects on nocturnal HPA axis activity in healthy controls. Blood samples were taken every 30 minutes, beginning at 1600 to 2400 on Days 1. At 1400 on Day 2, all subjects were given 0.5 mg of fludrocortisone. Blood was then resampled from 1600 to 2400 on Day 2 to assess nocturnal changes in HPA axis activity. Blood sampling was timed in the first half of the night to coincide with the time period of greatest activity of MR.

C. Statistics

Group averages, standard deviation and effect sizes are reported. The primary end-points were chosen to be mean cortisol and ACTH from 1600 to 2400 when greatest mineralocorticoid receptor activity is expected. To determine the differential effects of fludrocortisone, data from all subjects are pooled and the average values from 1600 to 2400 are computed. Next, mean values across this time interval are computed before (Time 1) and after (Time 2) fludrocortisone and the effect size for the difference between pooled mean values is computed.

II. Results:

A. Demographic Data

A total of 11 subjects met all inclusion/exclusion criteria and completed the study at the time of this interim study and analysis. Of these, 2 subjects were excluded from the analysis due to inadequate ACTH samples. Of the 9 HC subjects, there were 3 males and 6 females. The average age was 30.9 (10.8), ranging from 21 to 54.

B. Primary End-Points

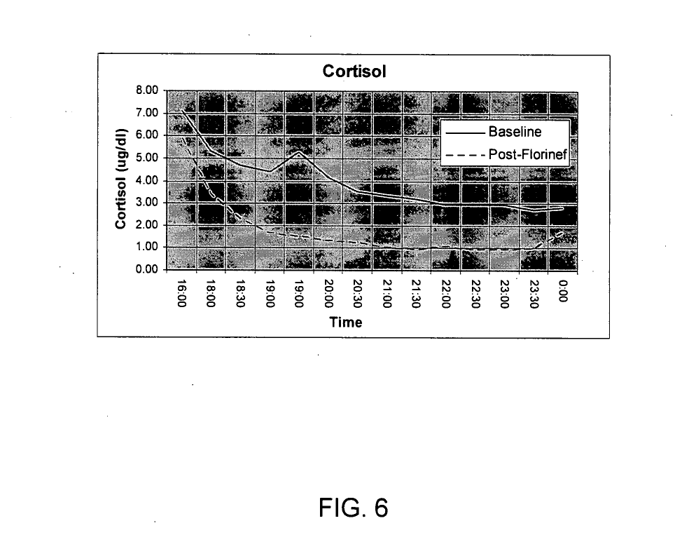

Cortisol. Average cortisol levels versus time are given in FIG. 6. Shown are baseline cortisol from 1600 to 2400 on the nights immediately before and after afternoon administration of fludrocortisone. Table 4 gives mean and standard deviation for both time periods. Relative to baseline, treatment with 0.5 mg fludrocortisone decreased mean cortisol during the interval from 1600 to 2400 from 3.99 ug/dl (1.283) to 1.77 ug/dl (1.38), for a difference of 2.19 ug/dl (p=0.003, effect size 1.65). This decrease coincides with a lowering of cortisol and ACTH at the nadir.

TABLE 4
Effect of Fludrocortisone on Mean Cortisol and ACTH Values

| | Difference | | Standard | AUC | Standard | | |
|---|---|---|---|---|---|---|---|
| | (Tx − Baseline) | AUC Baseline | Deviation | Treatment | Deviation | P value | Effect Size |
| Mean Cortisol | 2.19 | 3.96 | 1.283 | 1.77 | 1.38 | 0.003 | 1.65 |
| (1600 to 2400) | | | | | | | |
| Mean ACTH | 5.76 | 18.87 | 1.275 | 13.2 | 1.27 | 0.0000 | 4.46 |
| (1600 to 2400) | | | | | | | |
| Mean Cortisol/ACTH | 0.07 | 0.192 | 0.061 | 0.124 | 0.084 | 0.0686 | 0.92 |
| (1600 to 2400) | | | | | | | |

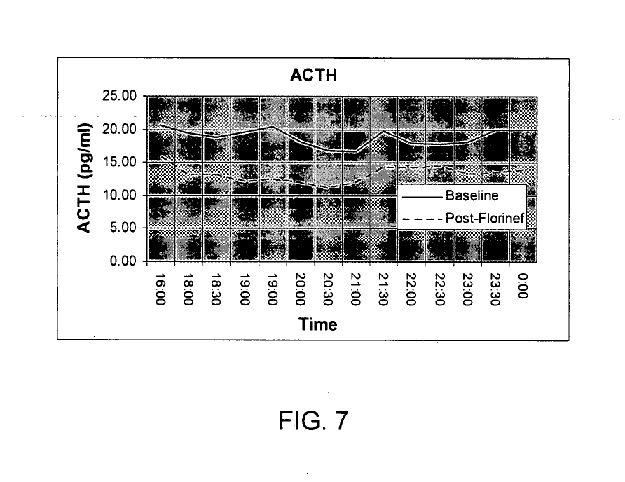

ACTH. Average ACTH levels versus time are given in FIG. 7. Shown are baseline ACTH from 1600 to 2400 on the nights immediately before and after afternoon administration of fludrocortisone. Again, Table 4 gives mean and standard deviation for both time periods. Relative to baseline, treatment with 0.5 mg fludrocortisone decreased mean ACTH during the interval from 1600 to 2400 from 18.87 pg/ml (1.275) to 13.2 pm/ml (1.27), for a difference of 5.76 pg/ml (p<0.0001, effect size 4.46).

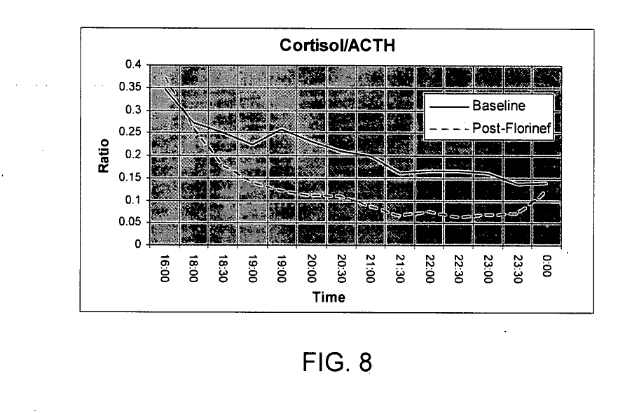

Post-Hoc Analysis Post-hoc analysis was performed to determine if fludrocortisone has a differential effect on the ratio of cortisol to ACTH concentration. FIG. 8 shows average ratios versus time before and after fludrocortisone. Mean of this ratio from 1600 to 2400 is computed again: Relative to baseline, treatment with 0.5 mg fludrocortisone decreased mean of the ratio cortisol/ACTH during the interval from 1600 to 2400 from 0.192 (0.061) to 0.124 (0.084), for a net decrease of 0.0686 (p=0.0058, effect size=0.92).

III. Discussion:

This pilot study evaluated acute effects of fludrocortisone on nocturnal HPA axis activity in healthy controls. Nocturnal HPA axis hyperactivity is associated with many conditions, including chronic insomnia (Rodenbeck and Hajak 2001; Vgontzas, Bixler et al. 2001; Buckley and Schatzberg 2005), depression and healthy aging (Born and Fehm 1998; Buckley and Schatzberg 2005). MR activity is greatest in the early nocturnal sleep period (Spencer, Kim et al. 1998) and plays an important role in decreasing the nocturnal nadir of cortisol. Nocturnal cortisol may be an indirect marker for nocturnal CRH activity.

Endogenous cortisol is an activator of MR and GR, and has greater affinity for MR than GR (Reul and de Kloet 1985). It has thus been assumed that MR's are fully occupied prior to GR's being occupied by cortisol. Along this traditional line of thinking, in a recent study of healthy and aged subjects. (Otte, Yassouridis et al. 2003), metyrapone was used to first unload glucocorticoid receptors and then the MR agonist fludrocortisone was administered. The nocturnal level of cortisol subsequently decreased. The ability of an MR agonist, alone, to additionally activate MR (above and beyond that from cortisol) and inhibit nocturnal HPA axis, to our knowledge, has not been reported. Herein, the MR agonist fludrocortisone was given without first “unloading” receptors with metyrapone and its effect on nocturnal HPA axis activity was measured. We predicted that, although endogenous cortisol may have greater affinity for MR than GR in brain, a direct MR agonist additionally would activate MR, above that of endogenous cortisol, to inhibit HPA axis activity. This inhibition of HPA axis activity would manifest in terms of decreased concentration of ACTH and cortisol. Since MR activity is greatest during the first part of the night and around the time of the -nadir, only the first part of the night was studied. We presumed that an MR agonist would inhibit HPA axis activity via hippocampal inhibition of the paraventricular nucleus via the nucleus basalis terminalis pathway. Alternatively, fludrocortisone may directly alter adrenal sensitivity to ACTH at the level of the adrenal gland.

Results show 0.5 mg fludrocortisone decreases mean cortisol from 1600 to 2400 with a large effect size of 1.65. Similarly, it also decreases mean ACTH from 1600 to 2400 with a large effect size of 4.46. Furthermore, the ratio of cortisol/ACTH also decreases during the same period, with an effect size of 0.92. Both the decrease in cortisol and ACTH are similar to those reported when fludrocortisones was given after “unloading” mineralocorticoid receptors by pre-treating with metyrapone (Otte, Jahn et al. 2003).

That fludrocortisone acutely decreases nocturnal cortisol and ACTH, suggests that it may be an agent for decreasing brain hypothalamic CRH. Furthermore, it suggests that even though endogenous cortisol has a greater affinity for MR than GR, MR is not always fully activated and is subject to agonist effects. This finding is significant in that the ability to alter HPA axis activity has useful clinical applications. For example, conditions associated with HPA axis hyperactivity include chronic insomnia, depression and healthy aging. Comorbid cognitive benefits are expected as well, given the known associations between excess glucocorticoid activity and cognitive function. Thus, an MR agonist such as fludrocortisone will have clinical applications in these populations.

Another interesting effect is the trend towards a reduction in the ratio of cortisol/ACTH towards the nadir which may represent a decrease in adrenal sensitivity to ACTH. We know that adrenal sensitivity changes across the 24 hour period, and is lowest at the time of the nocturnal nadir. Rat studies show that cortisol secretion is affected by a direct autonomic connection from the suprachiasmatic nucleus (SCN) to the adrenal cortex (Buijs, Wortel et al. 1999) as well as by splanchnic innervation of the adrenal gland (Ulrich-Lai, Arnhold et al. 2006). In the former case, cortisol is secreted without ACTH stimulation. In the latter case, splanchnic innervation influences adrenal sensitivity to ACTH. In both cases, the ratio of cortisol/ACTH is impacted. As such, the MR agonist fludrocortisone also decreases adrenal sensitivity to ACTH, in addition to its apparent inhibitory effect on CRH and ACTH at the level of the PVN. An alteration in adrenal sensitivity is consistent with an earlier study suggesting that an MR antagonist increased adrenal sensitivity to ACTH (Young, Lopez et al. 1998).

In summary, to our knowledge, this is the first study of the MR agonist fludrocortisone on nocturnal HPA axis activity, without first unloading the receptors with metyrapone. The effect size for a net decreased in mean cortisol and mean ACTH, from 1600 to 2400, is large. This indicates a significant impact of fludrocortisone on inhibiting nocturnal HPA axis activity. Furthermore, the fact that fludrocortisone can inhibit the axis without first depleting cortisol by pre-treatment with metyrapone, indicates that brain MR's are not fully occupied. The decrease in cortisol per ACTH indicates a role on adrenal sensitivity as well. The ability to lower nocturnal HPA axis activity has many useful clinical implications, including its benefit in insomnia, depression, healthy aging and other disorders associated with enhanced HPA axis activation.

It is evident from the above results and discussion that the subject invention provides for highly efficient methods and compositions for modulating the circadian rhythm of a subject, which can be employed in the treatment of individuals suffering from sleep disorders and in individuals desiring to adapt to different time schedules. As such, the present invention represents a significant contribution to the art.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method for modulating a circadian rhythm of a subject comprising:
administering to the subject an effective amount of at least one of glucocorticoid receptor antagonist, a CRH antagonist and an MR agonist to modulate the circadian rhythm in the subject.

2. The method of claim 1, wherein the modulating results in phase shifting the subject's sleep cycle.

3. The method of claim 1, wherein the method is a method for treating a subject for a sleep disorder.

4. The method of claim 3, wherein the sleep disorder is insomnia.

5. The method of claim 3, wherein the sleep disorder is Circadian Rhythm Sleep Disorder, Delayed Sleep Phase Type.

6. The method of claim 3, wherein the sleep disorder is Circadian Rhythm Sleep Disorder, Advanced Sleep Phase Type.

7. The method of claim 1, wherein the method is a method for treating Obstructive Sleep Apnea.

8. The method of claim 1, wherein the method is a method for treating inter-time zone travel induced sleep disturbance.

9. The method of claim 1 wherein the method is a method for treating Circadian Rhythm Sleep Disorder, Shift Work Type.

10. The method of claim 1 wherein the method is a method for treating Narcolepsy.

11. The method of claim 1, wherein said method comprises administering a glucocorticoid receptor antagonist to said subject.

12. The method of claim 11, wherein the glucocorticoid receptor antagonist is mifepristone.

13. The method of claim 11, wherein the glucocorticoid receptor antagonist is selected from the group consisting of RU009 and RU044.

14. The method of claim 1, wherein the subject is a mammal.

15. The method of claim 14, wherein the mammal is human.

16. The method according to claim 1, wherein said method is a method for modulating phase shifting of a sleep cycle of a mammalian subject.

17. The method of claim 16, wherein the method is a method for treating a subject for a Circadian Rhythm Sleep Disorder, Shift Work Type.

18. The method of claim 16, wherein the method is a method for treating a subject for inter-time zone travel induced sleep disturbance (Circadian Rhythm Sleep Disorder, Jet Lag Type).

19. The method according to claim 3, wherein said method further comprises identifying said subject suffering from a sleep disorder.

20. A kit comprising:
a glucocorticoid receptor antagonist; and
instructions for using the glucocorticoid receptor antagonist to treat a sleep disorder in a subject.
